(19) United States
(12) Patent Application Publication
(10) Pub. No.: US 2009/0041822 A1
(43) Pub. Date: Feb. 12, 2009

(54) TISSUE TRANSPLANT CONSTRUCT FOR THE RECONSTRUCTION OF A HUMAN OR ANIMAL ORGAN

(75) Inventors: Gouya Ram-Liebig, Dresden (DE); Oliver Hakenberg, Dresden (DE); Manfred Wirth, Dresden (DE)

Correspondence Address: BROUSE MCDOWELL LPA, 388 SOUTH MAIN STREET, SUITE 500, AKRON, OH, 44311, US

(73) Assignee: TECHNISCHE UNIVERSITAT DRESDEN, Dresden (DE)

(21) Appl. No.: 11/572,956

(22) PCT Filed: Jul. 18, 2005
(86) PCT No.: PCT/DE05/01266

§ 371 Date: May 30, 2008

(30) Foreign Application Priority Data
Jul. 30, 2004 (DE) 10 2004 037 184.9

(51) Int. Cl. A61L 27/24; A61L 27/56

(52) U.S. Cl. 424/422; 424/93.7

(57) ABSTRACT

The invention relates to a tissue transplant construct for the reconstruction of a human or animal organ, where the tissue transplant construct comprises:
a) a biologically compatible, collagen-containing membrane, and
b) one or more layers of organ-specific tissue cells on the membrane, and
   where the outermost layer of the organ-specific tissue cells is a layer of terminally differentiated organ-specific tissue cells.

The present invention relates to a tissue transplant construct for the reconstruction of a human or animal organ, as well as to a process for its preparation, and to the use of said tissue transplant construct. More especially, the present invention relates to a tissue transplant construct for the reconstruction of the urinary bladder.

It may be necessary to reconstruct the bladder in the case of various congenital or acquired diseases, such as for example myelomeningocele, bladder and cloacal dystrophy, injuries, malignant tumors, neuropathic dysfunctions and detrusor instability. The reconstruction, i.e. the replacement of damaged or diseased areas of a bladder, is effected with the aid of a tissue transplant construct, which is intended to serve as a matrix for the later growth of the original tissue.

It is mainly autologous gastrointestinal segments, obtained from the same person, that are currently used as matrices. Autologous de-epithelialized gut segments are an alternative, but they are less frequently used. However, these tissue transplant constructs have serious disadvantages. Thus, the use of gastrointestinal segments carries the risk of certain complications, such as for example electrolyte disturbances, mucus production, formation of calculi, and malignant degenerative processes. The reason for this is that the gastrointestinal mucosa is in constant contact with the urine. The use of de-epithelialized gut segments causes complications, such as the shrinkage of the segment.

Although cell-free matrices are suitable for the reconstruction of minor defects in the bladder, it is advantageous to coat the matrix if a larger defect is to be repaired [J. J. Yoo, J. Meng, F. Oberpenning and A. Atala: “Bladder augmentation using allogenic bladder submucosa seeded with cells”, Urology, 1998 February;51(2), pp. 221-225]. It has also been proposed to use urothelial cells for coating a matrix for bladder reconstruction. The aim here is to protect the matrix from the decomposing action of urine, since only urothelial cells can form a suitable barrier against urine and so protect the matrix from it, as well as from calcification and rejection.

It is also known that only terminally differentiated superficial cells of the urothelial layer ensure the required functional performance and in particular form a barrier against urine and its constituents. This is because the infiltration of toxic substances present in the urine is guaranteed by terminally differentiated urothelial cells, which are therefore essential for the maintenance of both the osmotic gradient in the bladder and tissue homeostasis.

Less differentiated urothelial cells are not as functional as terminally differentiated ones. Therefore, a tissue transplant construct made with a matrix coated with less well differentiated urothelial cells can lead e.g. to infections, calcification and matrix rejection, due to the functional inadequacy of such urothelial cells.

Using autologous urothelial cells would help to prevent rejection of the matrix coated with urothelial cells, but it presents certain problems, since a biopsy sample from an old and diseased bladder has been found to possess a rather limited regeneration potential [E. Y. Cheng and B. P. Kropp: Urologic tissue engineering with small intestinal submucosa: potential clinical applications, World J. Urol., 1998 February;51(2),pp. 26-30]. Furthermore, it is difficult to redifferentiate these urothelial cells to obtain normally functioning, highly differentiated cells, because the urothelial cells obtained in this way lose their characteristic in vivo phenotype during proliferation [Von der Zellkultur zum Tissue engineering (‘From Cell Culture to Tissue Engineering’), W. W. Minuth, R. Strehl and K. Schumacher, Pabst Science Publishers].

Viable autologous tissue constructs have been described by several authors [see refs. 1-5]. The limited regenerating ability of urothelial cells obtained from old and diseased bladders is known from reference 6. The loss of the characteristic phenotype of urothelial cells during in vivo proliferation is described in reference 7. The influence of extracellular matrices is discussed in detail in references 8-16.

The aim of the invention is to eliminate the disadvantages of the prior art, notably by providing a tissue transplant construct for the reconstruction of a human or animal organ, especially a urinary bladder, which construct possesses most of the same protective functional characteristics as healthy native urothelial tissue, and can therefore support the further generation of a muscular layer over the membrane after implantation. Another aim of the invention is to provide a process for the preparation of such a tissue transplant construct. A further aim of the invention is to use the tissue transplant construct in question.

These aims are achieved by means of the features specified in Claims 1, 17 and 29. Suitable embodiments of the invention will emerge from the features specified in Claims 2-16, 18-28, 30 and 31.

According to the invention, a tissue transplant construct for the reconstruction of a human or animal organ comprises a biologically compatible, collagen-containing membrane and one or more layers of organ-specific tissue cells on this membrane, where the outermost layer of the organ-specific tissue cells consists of terminally differentiated organ-specific tissue cells.

The term “organ-specific tissue cells” is used here to convey that the tissue cells that are to be applied to the membrane come from the same organ as the organ to be reconstructed, or from an organ identical to it. For example, if the aim is to reconstruct a bladder, then the organ-specific tissue cells in question are urothelial cells in the present case.

Organs are functional units of the body, and the preferred example in the present context is the bladder.

In a preferred embodiment, the tissue transplant construct for bladder reconstruction comprises a biologically compatible, collagen-containing membrane and one or more layers of urothelial cells on this membrane, where the outermost layer of the urothelial cells consists of terminally differentiated urothelial cells.

If the membrane carries only one layer of tissue cells, then this layer is considered to be the outer layer which is a layer of terminally differentiated tissue cells.

The outermost layer consists of terminally differentiated tissue cells, while the other layers are composed of non-terminally differentiated tissue cells.

Unless otherwise specified, the terms “membrane” and “matrix” are used here interchangeably. The membrane should contain the components of the extracellular matrix (ECM), especially its growth factors, and is preferably a biological membrane.

The term “collagen-containing membrane” is used here to denote a membrane with a collagen base.

The term “reconstruction” is used here in the sense of replacing damaged or diseased areas of a human or animal organ, especially of a bladder.

The term “stromal induction” is used here to denote the kind of tissue cell proliferation that is induced by the stroma and preferably by fibroblasts. Stromal induction should lead to a sufficient proliferation of the tissue cells to cover fairly large areas (up to 40 cm²) of the membrane surface.

The number of tissue cell layers formed on the membrane is 1-7, preferably 2-7 and especially 4-7, where the outermost layer consists of terminally differentiated tissue cells. If the construct is to be used for reconstructing a human organ, the tissue cells are preferably autologous human tissue cells, but preferably only the outermost layer consists of terminally differentiated tissue cells.

Cellular communication within the tissues and organs is ensured by the complex molecular structure of the extracellular matrix (ECM). The biochemical and biophysical signals sent out by the extracellular matrix are fundamental e.g. for regulating the cellular activities, such as adhesion, morphogenesis, migration, proliferation, differentiation and function creation. The role of the extracellular matrix and its interaction with cells are very important for development, regeneration and pathogenesis in the case of the bladder as well. For example the age-dependent factors cause changes in the ultrastructure of the extracellular matrix thus and in the regeneration potential of the bladder tissue.

The biologically compatible, collagen-containing membrane is preferably a mammalian tissue, especially a human or hog (porcine) submucosal or cutaneous tissue, whose preparation is well known.

Therefore, the first prerequisite for successful cell expansion and the differentiation of epithelial cells is the correct choice of the membrane, with the right constituents of the extracellular matrix and the specific signalling processes of these constituents as regards their orienting and regulating function.

When preparing an acellular membrane, the important growth factors and other constituents of the extracellular matrix should not therefore be completely removed, so as to ensure the most natural medium possible for the cell culture and to facilitate the dynamic and two-way interactions between the matrix constituents and the urothelial cells applied to the matrix.

The biologically compatible, collagen-containing membrane is preferably an intestinal submucosa, an acellular bladder submucosa or an acellular dermal matrix.

The following are particularly suitable for use as biologically compatible, collagen-containing membranes.
Small intestinal submucosa (SIS), derived from the hog's small intestine. This contains a combination of various types of collagen, glycoproteins, proteoglycans and functional growth factors, which play a role in cell migration, growth and differentiation [see ref. 15]. The almost acellular lyophilized form of this membrane can be obtained from Cook Biotech in Mönchengladbach, Germany under the name of lyophilized small intestinal submucosa (LSIS). The non-lyophilized form of SIS, called non-lyophilized small intestinal submucosa (NLSIS), can be obtained—like acellular bladder submucosa (ABS) and acellular dermal matrix (ADM)—by applying first an enzymatic treatment, and then a chemical treatment in an acellular manner, in order to minimize the immunogenicity after the transplantation of the construct formed with this base.
Acellular bladder submucosa (ABS), derived from animal or human bladder submucosa. This is an organ-specific matrix, which permits the physiologic generation of bladder tissue before and after transplantation.
Acellular dermal matrix (ADM), derived from skin. This can only be prepared from autologous tissue and is therefore free of any risk of infections being transmitted from donor to recipient. If it is used jointly with autologous serum, a fully autologous system is obtained, i.e. one that does not contain any fetal serum or an allogenic or xenogenic membrane.

The tissue transplant construct according to the invention can be prepared by a process that consists of the following steps:
(a) application of organ-specific tissue cells to a biologically compatible, collagen-containing membrane,
(b) proliferation of the applied organ-specific tissue cells in a culture medium under the influence of stromal induction, with the formation of one or more layers of organ-specific tissue cells on the membrane, and
(c) terminal differentiation of the outermost layer of the expanded organ-specific tissue cells under the influence of further stromal induction, by reducing the amount of mitogenic factors in the culture medium.

Accordingly, a tissue transplant construct for bladder reconstruction can be prepared by a process comprising the following steps:
(a) application of urothelial cells to a biologically compatible, collagen-containing membrane,
(b) proliferation of the applied urothelial cells in a culture medium under the influence of stromal induction, with the formation of one or more layers of urothelial cells on the membrane, and
(c) terminal differentiation of the outermost layer of the expanded urothelial cells under the influence of further stromal induction, by reducing the amount of mitogenic factors in the culture medium.

Reducing the amount of mitogenic factors means here the reduction of the serum concentration (e.g. the concentration of bovine embryo serum or autologous serum), while keeping the concentration of other stromal nutritive substances constant.

Steps b) and c) are carried out to effect first the expansion of the tissue cells (e.g. the urothelial cells), while retaining their epithelial phenotype, then to effect their differentiation, and finally to bring about the terminal differentiation of the outermost layer of the tissue cells. The culture medium must therefore be chosen such that the membrane selected in conjunction with the appropriately adapted culturing conditions will induce first the migration and division of the cultured cells, and then their functional maturation in order to permit the development of a highly differentiated functional urothelial construct in bladder reconstruction.

For this purpose, the tissue cells applied to the membrane in step a) must be cultured in a medium containing bovine embryo serum (BES) in order to effect their proliferation and differentiation, as well as the terminal differentiation of the outermost layer. It is particularly advantageous to culture the tissue cells applied to the membrane in step (a) for proliferation and terminal differentiation in a fibroblast-conditioned medium (FBCM) that contains some bovine embryo serum (BES).

The culture medium preferably contains 5-10% of bovine embryo serum (BES).

After step (b), the concentration of the bovine embryo serum (BES) should be reduced in order to effect the terminal differentiation of the superficial cells after the sufficient expansion of the tissue cells on the membrane. The concentration of the bovine embryo serum (BES) is preferably reduced here by 50% and especially from 5-10% to 0-1%.

“Sufficient expansion” means here that the membrane is fully covered with a number of layers of tissue cells.

To prepare the tissue transplant construct for medical use, the process includes the preparation of a tissue transplant construct for bladder reconstruction, in which process urothelial cells are first obtained from small bladder biopsy samples and are then subjected to a (first) expansion under conventional conditions. The urothelial cells are then applied to a biocompatible membrane with a collagen base. After sufficient further expansion under the influence of stromal induction, the urothelial cells are made to undergo terminal differentiation under stromal induction by reducing the amount of mitogenic factors.

The tissue transplant constructs according to the invention are constructs that correspond to the in vivo phenotype, owing to the terminal differentiation of the superficial cell layer, and thus they are like native tissue in their function, being specifically like native urothelial tissue in the case of a bladder. It has thus become possible for the first time to obtain functioning urothelial constructs that adequately protect the biological membrane from urine. By using a culture medium conditioned with autologous fibroblasts, it is possible to dispense with xenogenic feeder layers and other commercially available additives, such as the epidermal growth factor (EGF). This has financial advantages and allays the fears about toxicity, carcinogenicity and the transfer of diseases. The bovine embryo serum (BES) can be replaced by autologous serum, in which case the culture medium does not contain any xenogenic additives.

Autologous serum can be obtained in the usual way from the blood of the patient who is to be treated.

The invention is explained below in more detail with the aid of examples and the drawings, where:

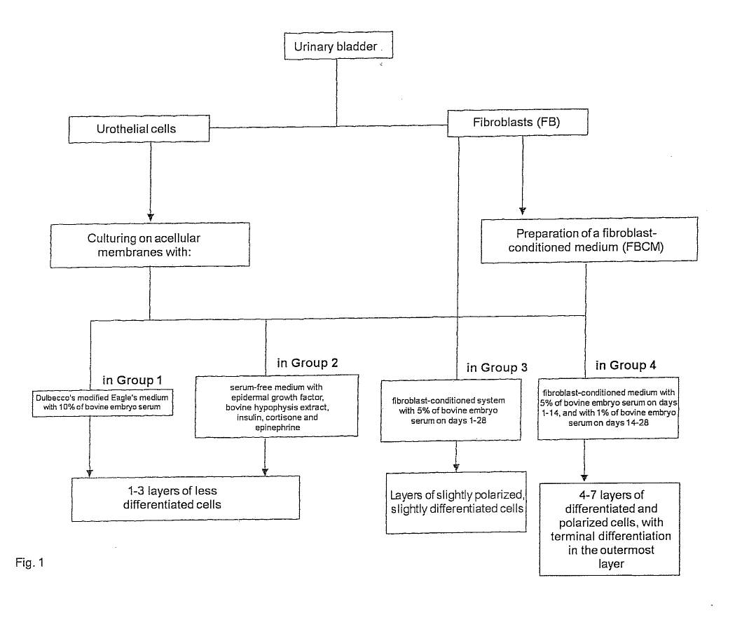

FIG. 1 shows four methods 1-4 that can be used to effect proliferation and terminal differentiation,

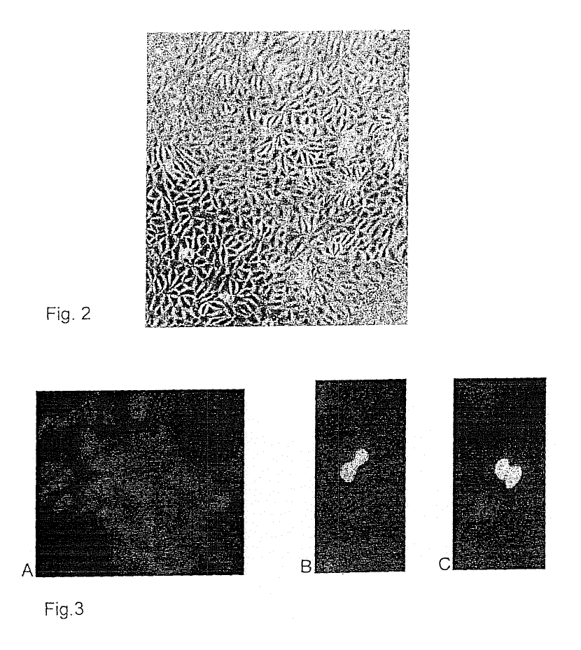

FIG. 2 is a phase-contrast micrograph of a monolayer from porcine bladder mucosa,

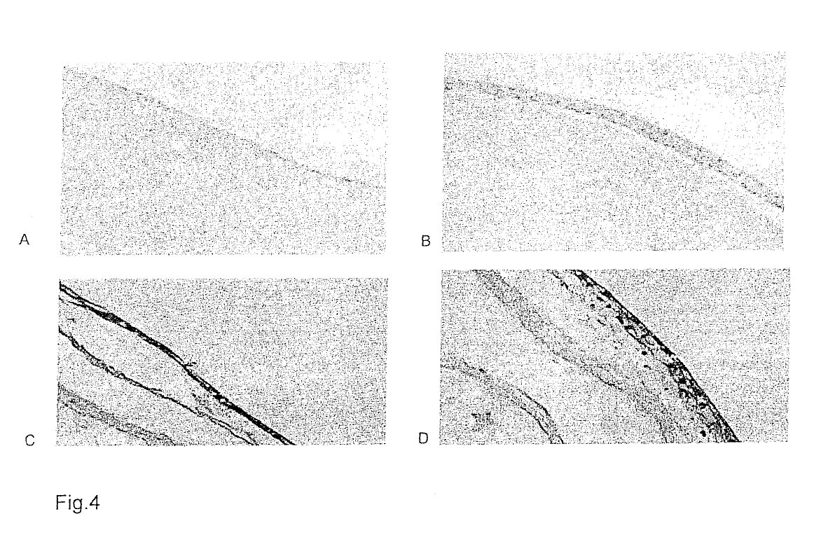

FIG. 3 shows a porcine bladder construct on lyophilized small intestinal submucosa (LSIS) that has been stained with DAPI, where FIG. 3a shows the visible multilayers of the cell nuclei on the membrane, and FIG. 3b and FIG. 3c show the mitotic activity of the dividing nuclei of cultured cells on lyophilized small intestinal submucosa (LSIS),

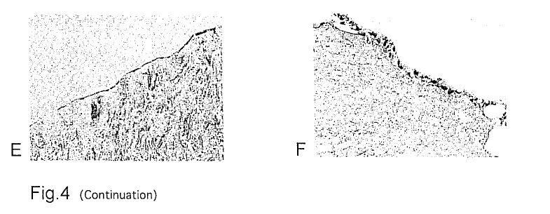

FIG. 4 shows some urothelial cell constructs on acellular membranes without any fibroblast induction, embedded in paraffin wax, where FIGS. 4A and 4B were obtained with hematoxylin-eosin staining on acellular bladder mucosa (ABS) ; FIGS. 4C and 4D were obtained by immunohistochemical treatment with pancytokeratin on lyophilized small intestinal submucosa, and FIGS. 4E and 4F were obtained by hematoxylin-eosin staining on acellular dermal matrix (ADM),

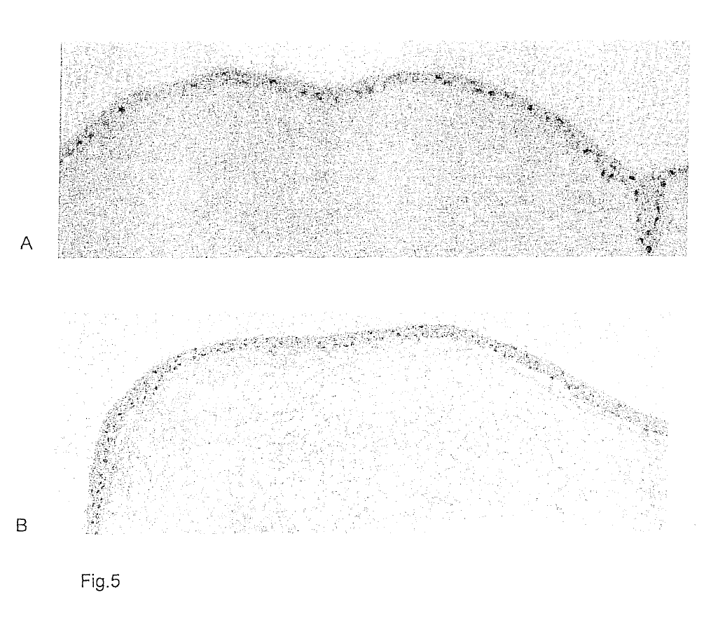

FIG. 5 shows a histological comparison of the urothelial cells attached to and growing on acellular bladder submucosa, where FIG. 5A shows urothelial cells cultured without fibroblast induction, and FIG. 5B shows urothelial cells cultured with fibroblast induction,

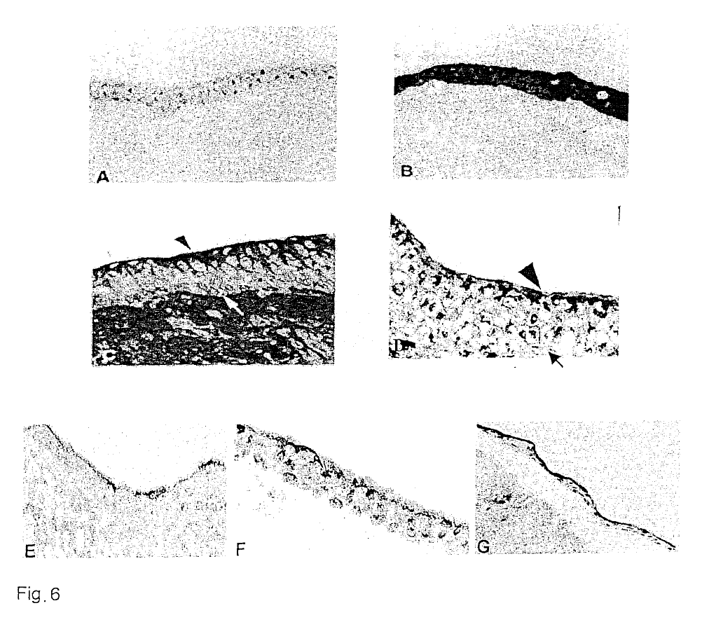

FIG. 6 shows the histologic, histochemical and immunohistochemical assays of porcine and human urothelial cell constructs on acellular bladder submucosa (ABS), where FIGS. 6A, 6B and 6D show the situation after the bovine embryo serum concentration was reduced from 5% to 1%, FIGS. 6C and 6E show the native porcine urinary bladder, FIG. 6F shows the porcine urothelial cell construct, and FIG. 6G shows a human urothelial cell construct,

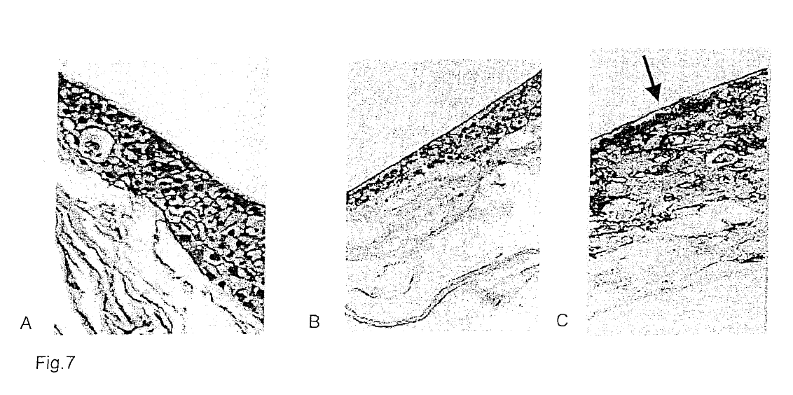

FIG. 7 shows the histologic, histochemical and immunohistochemical assays of porcine and human urothelial cell constructs on lyophilized small intestinal submucosa (LSIS), where FIG. 7A was obtained with hematoxylin-eosin staining; FIG. 7B was obtained with AE1/AE3 staining, and FIG. 7C was obtained by staining with wheat germ agglutinin (WGA),

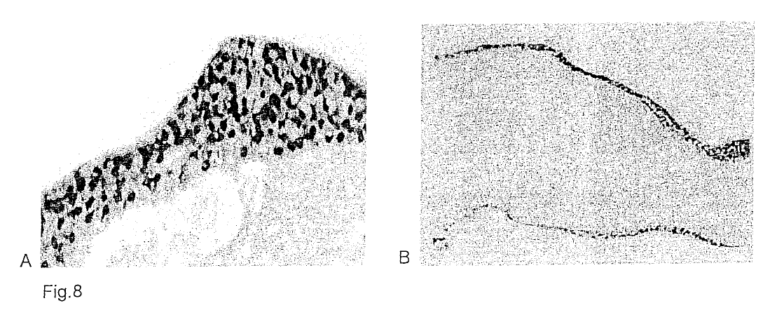

FIG. 8 shows some urothelial cell constructs on non-lyophilized small intestinal submucosa (NLSIS), embedded in paraffin wax, where the bovine embryo serum (BES) concentration was kept constant during the culturing period, the magnification being by a factor of 40 in FIG. 8A and by a factor of 10 in FIG. 8B, and

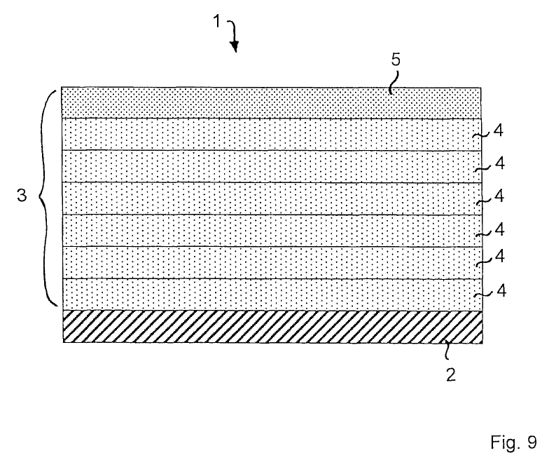

FIG. 9 is a schematic representation of a urothelial cell construct according to the invention.

### EXAMPLES

The following examples serve to elucidate the process for the preparation of bladder tissue transplant constructs according to the invention.

Unless otherwise specified, the abbreviations used here have the following meanings:
BCECF=2′,7′-bis-(2-carboxyethyl)-5- and -6-carboxyfluorescein
DAPI=4′,6′-diamidino-2-phenylindole
DMEM=Dulbecco's modified Eagle's medium
ECM extracellular matrix
EGF=epidermal growth factor
FB=fibroblast
BES=bovine embryo serum
KGM=keratinocyte growth medium
UC=urothelial cells
PBS=phosphate-buffered physiological saline
PI=propidium iodide
WGA=wheat germ agglutinin
FBCM=fibroblast-conditioned medium.

### Materials and Methods

### Biological Acellular Membranes

Lyophilized small intestinal submucosa (LSIS) can be obtained commercially from Cook Biotech in Mönchengladbach, Germany. To prepare non-lyophilized small intestinal submucosa (NLSIS), acellular bladder submucosa (ABS) and cellular dermal matrix (ADM), the corresponding porcine samples were treated for 48 hours with 1% of Triton X-100 from Sigma-Aldrich in Taufkirchen, Germany.

The membranes had a size of up to 40 cm² in each case.

### Urothelial and Fibroblast Cell Cultures

Bladder segments were taken from eight sacrificed hogs, because their bladder is similar to the human bladder [see ref. 15]. The human tissue samples were taken from the bladders of four adults who had undergone bladder surgery.

The mucosal and submucosal tissues were comminuted separately, digested with collagenase B in an amount of 200 u/ml (from Worthington Biochemical, Lakewood, N.J., USA), isolated by centrifugation at 2000 rpm, and cultured in a humid atmosphere consisting of 95% air and 5% carbon dioxide. The urothelial cells and the fibroblasts were in each case cultured in a supplemented keratinocyte growth medium (KGM) from CellSystems Biotechnologie, St. Katharinen, Germany and in Dulbecco's modified Eagle's medium (DMEM) from Invitrogen, Karlsruhe, Germany, to which 5-10% of bovine embryo serum (BES) had been added. The material obtained was then subjected to immunocytochemical assays with AE1/AE3 antibodies in a 1:150 concentration, Vimentin in a concentration of 1:100 and α-actin from smooth muscles in a concentration of 1:100, obtained from Dako, Glastrup, Denmark.

Fixed numbers of urothelial cells were applied to the surface of the membrane layers, using 10⁵ urothelial cells per cm², and the coated membranes were treated twice a week for up to 28 days by one of the following four methods 1-4.
(1) In Group 1 (controls), the urothelial cells were cultured in Dulbecco's modified Eagle's medium (DMEM), supplemented by 10% of bovine embryo serum (BES).
(2) In Group 2 (controls), the urothelial cells were cultured in a serum-free medium, supplemented by epidermal growth factor (EGF), bovine hypophysis extract (BHE), hydrocortisone, transferrin, insulin and epinephrine in the concentration recommended by the manufacturer.
(3) In Group 3 (controls), the fibroblasts were cocultured in Dulbecco's modified Eagle's medium (DMEM) with 5% of bovine embryo serum (BES) at the bottom of a Transwell culture insert.
(4) In Group 4, the urothelial cells were cultured in a fibroblast-conditioned medium (FBCM), supplemented by 5% of bovine embryo serum (BES) in the first 14 days and by 1% of bovine embryo serum (BES) in the next two weeks.

FIG. 1 shows the corresponding four methods 1-4. The urothelial cells and the fibroblasts had been harvested from a bladder by enzymatic treatment. After a (first) expansion, the urothelial cells were cultured on the specified membranes, using the media specified for Methods 1-4. The fibroblast-conditioned culturing system used in Groups 3 and 4 gave more cell layers than those not conditioned with fibroblasts (Groups 1 and 2). The reduction in the mitogenic stimuli in Group 4 after day 14 induced a high degree of differentiation in the urothelial cells in the same space of time.

The media used in all four methods 1-4 were first supplemented by 0.09 mmol of calcium. A high calcium concentration of 2.5 mmol was ensured at the air-liquid interface of the culture medium in order to promote layer formation.

Medium Conditioned with Bladder Fibroblasts

After primary culturing, 10⁶ fibroblasts were transferred into a 75 m³ cell culture flask and cultured on Dulbecco's modified Eagle's medium (DMEM), supplemented by 5% or 1% of bovine embryo serum (BES). When a 90% confluence had been obtained, the media were changed, collected after 24 h, filtered on a filter (Falcon BD, Heidelberg, Germany), and stored at 4° C.

### Cellular Adhesion, Proliferation and Viability Tests

To determine the surface density of the cultured cells on the membranes, the cell count obtained after separation from the culture dish was subtracted from the cell count found in the suspension 2 and 10 hours after seeding them on the membrane. The proliferation assay was carried out with 4′,6-diamidino-2-phenylindole (DAPI) on days 2, 14 and 28 after application. The vitality assays were performed 6 hours, 14 days and 28 days later with 2′,7′-bis-(2-carboxyethyl)-5- and -6-carboxyfluorescein (BCECF) and propidium iodide (PI), both obtained from Mobitec (Göttingen, Germany), and both used in a concentration of 5 μmol after incubation for 20 minutes.

### Histologic, Histochemical and Immunohistochemical Staining of the Cultured Cells on the Membranes

The material was fixed for 24 hours in 4% formaldehyde buffered to a neutral pH, and then embedded in paraffin wax. The sections were either stained with hematoxylin-eosin, or else wheat germ agglutinin (WGA) was used for immunohistochemical examination and lectin staining. The primary antibodies to pancytokeratin (AE1/AE3 from Dako) were diluted in a ratio of 1:150 after treating the preparation with pronase. The detection of the primary antibodies was accomplished with the aid of either a horseradish peroxidase system (LASB system from Dako) or an alkaline phosphatase detection system (Vectastain ABC kit from Vector Laboratories, Grüinberg, Germany). The dye substrate was therefore either diaminobenzidine, which is brown, or alkaline phosphatase neo-fuchsin, which is red. Lectin wheat germ agglutinin (from Vector Laboratories) was diluted in a ratio of 1:100, and the Vectastain ABS kit was used for further treatment. Immuno-staining with anti-uroplakin III antibodies, obtained from Progen in Heidelberg, Germany, was carried out at a 1:10 dilution and developed with the LSAB kit from Dako according to the manufacturer's instructions.

### Results

### Conventional Cell Cultures

Primary cultures of fibroblasts and urothelial cells were harvested from small biopsy specimens from porcine and human bladder cells (see FIG. 2). Their epithelial nature was confirmed by urothelial cell staining, using antibodies to cytokeratin with a broad reactivity. The fibroblasts stained positive with Vimentin and did not react with the pancytokeratin antibodies. Both cell types showed a negative marking for α-actin of smooth muscles.

FIG. 2 shows a picture taken under a phase-contrast microscope at a tenfold magnification of porcine urothelial cells harvested from porcine bladder mucosa by micro-dissection. The cells showed their polygonal phenotype in the two-dimensional, serum-free culturing system.

The strongly supplemented keratinocyte growth medium (KGM) is known to be suitable for maintaining the proliferation potential of the urothelial cells in vitro. However, its large amount of growth factors and additives may cause problems when determining the regulating factors of a fibroblast origin, located on the urothelial cells. This is why not only this medium was used (in Group 2), but also a universal medium (Dulbecco's modified Eagle's medium with bovine embryo serum—DMEM/BES) in another experimental series to improve the determination of the interactions between the urothelial cells and the extracellular matrix (ECM in Group 1) and the interactions between the urothelial cells, the fibroblasts (FB) and the extracellular matrix (ECM) by indirect coculturing (in Group 3) as well as by the adjustment of the fibroblast-conditioned medium (FBCM in Group 4).

### Adhesion of the Cells to the Membranes

Two hours after seeding, the degree of adhesion of the cells was as follows: 80% in the case of acellular bladder submucosa (ABS) and non-lyophilized small intestinal submucosa (NLSIS), 70% in the case of lyophilized small intestinal submucosa (LSIS), and 60% in the case of acellular dermal matrix (ADM). After 10 hours, the final cell count in the suspension indicated that the number of cells remaining in the solution was as follows: none with NLSIS and ABS, 10% with LSIS, and 20% with ADM. When porcine and human urothelial cells were cultured in non-conditioned media and without the addition of bovine embryo serum (BES) and growth factors, the adhesion of the cells was low, since these cells died off in the first few days (not shown) and therefore could not be taken into account later.

### Vitality of the Cells

The cells retained a high degree of vitality (=80%) in all the groups over the 28-day period. When double staining was used on the urothelial cell cultures, the cells showed a green fluorescence due to metabolized BCECF-AM. The urothelial cells built up on the surface of the membranes survived and remained viable after 6 hours. They formed coalescing layers on the whole surface of the membranes, which were in evidence on days 14 and 28. Up to day 14, hardly any dead cells (<5%) were seen in any of the groups. The porcine and human urothelial cells receiving a fibroblast induction contained a greater number of viable cells than the control groups 1 and 2, judging by the more intense green fluorescence from several layers (not shown). In Group 4, fewer than 10% of the cells were dead on day 28, while in Group 3, 20% of them were dead on the membrane.

### Proliferation of the Cells

The DAPI-stained cell nuclei exhibited various stages of mitosis without any detectable apoptosis (<5%) on days 2 and 14 in all four groups (see FIG. 3). The multilayers of nuclei were considerably richer in the case of cultures with fibroblast induction up to day 28 (Groups 3 and 4). From day 2 to day 14, the mitotic index for the culture rose by a factor of 1.7 in the case of porcine cells and 1.3 in the case of human cells (for the controls in Groups 1 and 2), and by a factor of 3.2 for porcine cells and 2.9 for human cells in Groups 3 and 4. Hardly any proliferating nuclei were observed in Group 4 on day 28, but nuclei in various stages of mitosis were still in evidence in Group 3.

FIG. 3 shows a DAPI-stained porcine bladder construct based on lyophilized small intestinal submucosa (LSIS) at a magnification of 40. The nuclei were detected by indirect immunofluorescence 14 days after seeding. FIG. 3A shows multilayers of cell nuclei on the membrane. The mitotic activity of the dividing nuclei of the cultured cells on lyophilized small intestinal submucosa (LSIS) is shown in FIG. 3B and FIG. 3C, which are fluorescence micrographs taken at a magnification of 40.

### Histologic, Histochemical and Immunohistochemical Assays of the Constructs

The analysis of coalescing cultures showed cells with a positive response to pancytokeratin antibodies. The native tissues and the porcine and human urothelial cell constructs were also subjected to staining with wheat germ agglutinin and antibodies to uroplakin III.

The histologic assay showed that the urothelial cells were able to adhere to all four types of membrane, as well as to migrate and proliferate. In the controls of Groups 1 and 2, the cells migrated on day 1 from small groups to one or two layers, which ensured that by day 28 the whole surface of the membrane was increasingly covered with one or two layers in the case of lyophilized small intestinal submucosa (LSIS), and with two or three layers in the case of non-lyophilized small intestinal submucosa (NLSIS) and acellular bladder submucosa (ABS) (FIG. 4). In another experiment, the urothelial cells were cocultured on membranes, but the fibroblasts were separated off with the aid of a Transwell culture insert (Group 3). In the third experimental series, the epithelial cells were cultured on membranes in media conditioned with fibroblasts (Group 4). Urothelial cells cultured in media containing 10% of bovine embryo serum (BES, Group 1) or epidermal growth factor (EGF) and other standard additives (Group 2) showed a limited migration and membrane coverage, less good growth characteristics, lower proliferation rates and little differentiation in comparison with cultures involving fibroblast induction. They also showed a gradual aging after 4 weeks. Neither the bovine embryo serum (BES) nor the epidermal growth factor (EGF) and other exogenic additives induced either layer formation or terminal differentiation in the porcine and human urothelial cells (Groups 1 and 2) under the conditions specified here.

FIG. 4 shows some urothelial cell constructs on acellular membranes without any fibroblast induction, all embedded in paraffin wax, where FIGS. 4A, 4B, 4E and 4F were obtained with hematoxylin-eosin staining, and FIGS. 4C and 4D were obtained by immunohistochemical treatment with pancytokeratin. The urothelial cells on the acellular bladder submucosa (ABS in FIG. 4A), the lyophilized small intestinal submucosa (LSIS in FIG. 4C) and the acellular dermal matrix (ADM in FIG. 4E) all showed a monolayer of flat cells the day after seeding. The cells continued to proliferate and after 28 days had a rather cubic shape on the acellular bladder submucosa (ABS, see FIG. 4B), the lyophilized small intestinal submucosa (LSIS, see FIG. 4D) and the acellular dermal submucosa (ADM, see FIG. 4F). On day 28, the cultures consisted of two or three layers of cells on the acellular bladder submucosa (ABS, see FIG. 4B), and one or two layers of cells on the lyophilized small intestinal submucosa (LSIS, see FIG. 4d) and on the acellular dermal submucosa (ADM, see FIG. 4f). The epithelial origin of the urothelial cells was confirmed by the immuno-staining of the reconstructed urothelium in vitro, using pancytocreatin on the lyophilized small intestinal submucosa (LSIS, see FIGS. 4C and 4D).

The culture medium could be optimized by the addition of soluble nutrients, independently of fibroblasts (Groups 3 and 4). A culture of urothelial cells on matrices with fibroblasts, separated off with the aid of the Transwell culture insert, gave similar results as regards the morphology of the urothelial cells, layer-formation and differentiation when compared with the results of experiments with fibroblast-conditioned culture media.

These two experimental series involving Groups 3 and 4 showed more urothelial cell layers developing on the membrane surface in the same space of time than the urothelial cell cultures without fibroblast induction (in Groups 1 and 2, see FIG. 5). The hematoxylin-eosin staining in fact revealed an increasing migration of the urothelial cells from small groups to one, two or three layers (see FIG. 3) as opposed to 3-7 layers (in Group 4) or more layers (in Group 3, see FIGS. 6-8), which on day 28 fully covered the membrane surfaces having various sizes of up to 40 cm².

FIG. 5 shows a histologic comparison, at a magnification of 40, of the urothelial cells adhering to acellular bladder submucosa (ABS) and growing on it. These cells were cultured either without fibroblast induction (i.e. in a non-conditioned medium, see FIG. 5A) or with fibroblast induction (i.e. in a conditioned medium, see FIG. 5). A relatively low calcium concentration (0.09 mmol) was used, and 5 days after seeding, the cells covered the membrane with one or two layers in the non-conditioned medium (see FIG. 5) and with two or three layers in the medium with fibroblast induction (see FIG. 5B).

After proliferation accelerated by the fibroblast induction in Groups 3 and 4, the cells on day 14 had not reached terminal maturity with the high calcium concentration of 2.5 mmol. To induce terminal differentiation, the concentration of bovine embryo serum (BES) was reduced from 5% to 1% after the proliferation stage (14 days) in Group 4. This reduced the concentration of the mitogenic factors, while the concentration of the other nutrient components remained constant, with a downward regulation of the proliferating state of the cells. Under these conditions, the porcine and human cells showed full differentiation on the surface, as indicated by the results of histologic, histochemical and immunohistochemical assays, illustrated in FIGS. 6 and 7. This state was maintained to day 28. The intense staining of the superficial cells in the urothelial cell construct with wheat germ agglutinin (WGA), in addition to their strong expression of uroplakin III, a typical marker for urothelial cell differentiation, also point to a terminal differentiation of these cells (see FIGS. 6 and 7).

FIG. 6 shows a histologic assay (see FIG. 6A), a histochemical assay (see FIGS. 6C and 6D) and an immunohistochemical assay (see FIGS. 6B, 6E, 6F and 6G) of porcine and human urothelial cell constructs on acellular bladder submucosa (ABS, in FIGS. 6A, 6B and 6D) after reducing the bovine embryo serum (BES) concentration from 5% to 1%, in comparison with native porcine bladder (see FIGS. 6C and 6E). The hematoxylin-eosin staining of the urothelial cells cultured on acellular bladder submucosa (ABS, see FIG. 6A) shows a layered neo-urothelium obtained 28 days after seeding. The cultured urothelial cells exhibit a strongly positive reaction with pancytokeratin antibodies, indicated by a dark brown stain with the dye substrate diaminobenzidine (see FIG. 6B). The wheat germ agglutinin (WGA) staining of the urothelial cell construct (see FIG. 6D) is very similar to that of the native urothelium (see FIG. 6C) in the case of fibroblast induction. Wheat germ agglutinin predominantly stains the highly differentiated layers of native urothelium (see the head of the arrow in FIG. 6C) and includes the surface layer of the urothelial cell construct in vitro (see the head of the arrow in FIG. 6D). The less differentiated cells are not stained by wheat germ agglutinin (see the arrows). The staining with anti-uroplakin III antibodies, which produce a brown color, indicates a close similarity between the native porcine urothelium (see FIG. 6E), the porcine urothelial cell construct (see FIG. 6F), and the human urothelial cell construct (see FIG. 6G). As mentioned before, the intense expression of uroplakin III can only be seen in the surface layer of the native urothelium (see FIG. 6E) and only in the surface layer of the urothelial cell constructs (see FIGS. 6F and 6G, using a 40-fold magnification in FIGS. 6A to 6F, and a ten-fold magnification in FIG. 6G).

FIG. 7 shows the histologic, histochemical and immunohistochemical assays of urothelial cell constructs on lyophilized small intestinal submucosa (LSIS) with fibroblast induction on day 28. After the reduction of the bovine embryo serum (BES) concentration from 5% to 1%, hematoxylin-eosin staining revealed up to seven layers of morphologically differentiated cells (see FIG. 7A). The positive staining of the urothelial cells with AE1/AE3 antibodies confirmed the epithelial origin of these cells (see FIG. 7B). The staining with wheat germ agglutinin (WGA, see FIG. 7C) indicated that the superficial cells of the urothelial cell constructs were stained most intensely with lectin (see the arrow) when compared with the less differentiated cells in the lower layers (see FIG. 7B taken at a 10-fold magnification and FIGS. 7A and 7C taken at a magnification of 40).

The reduction in the amount of bovine embryo serum (BES), the epidermal growth factor (EGF), and the bovine hypophysis extract (BHE) when using a non-conditioned medium (Groups 1 and 2) led to non-proliferating cells, which gradually died off. In Group 3, the bovine embryo serum (BES) concentration was kept at 5% for 28 days. The migration and proliferation stages were maintained, leading to the expansion of the cells on the other side of the membrane and to layer formation of morphologically less polarized and weakly differentiated cells (see FIG. 8). Confirming the different proliferation stages of the neo-urothelium in Group 4, the demonstration of the activity of cell nuclei with the aid of DAPI staining mostly showed mitotic nuclei in the proliferative stage during the culturing period (on day 14, with 5% of bovine embryo serum), while the number of dividing nuclei was significantly lower in the terminally differentiated urothelium (on day 28, with 1% of bovine embryo serum).

FIG. 8 shows a urothelial cell construct on non-lyophilized small intestinal submucosa, embedded in a layer of paraffin wax. The bovine embryo serum (BES) concentration in the highly proliferating culture system was kept constant at 5% for 28 days, with fibroblast induction. In this culturing system, the cells continued to proliferate and showed a decrease in differentiation. They divided further and migrated to the other side of the membrane, so that the non-lyophilized small intestinal submucosa was covered with numerous layers of non-polarized cells, giving the picture of a hyperproliferative urothelium (FIG. 8A was taken at a magnification of 40, and FIG. 8B at a magnification of 10).

### Evaluation of the Results

Small intestinal submucosa (SIS), acellular dermal matrix (ADM) and acellular bladder submucosa (ABS) all supported the growth of the urothelial cells, which made the interactions between cells and the extracellular matrix (ECM) possible, together with the formation of two or three layers of urothelial cells in strongly supplemented culture media (in Groups 1 and 2). Acellular bladder submucosa (ABS) and non-lyophilized small intestinal submucosa (NLSIS) have about the same ability to support the proliferation and differentiation of urothelial cells. The small difference between lyophilized and non-lyophilized small intestinal mucosal membranes is due to the lyophilized form of small intestinal submucosa (SIS) [see ref. 18]. The acellular dermal matrix (ADM) also showed a good proliferation and differentiation of urothelial cells.

It has been reported that a serum-free medium with growth-stimulating additives is critical for the growth of urothelial cells under conventional culturing conditions [see ref. 19]. The addition of exogenic factors or 10% of bovine embryo serum (BES) in the control groups showed a moderate inducing effect on the urothelial cells, cultured on the membranes (in Groups 1 and 2).

To improve the culturing conditions of urothelial cells further, the inducing action of fibroblasts from the lamina propria of urothelial cells was employed, which supplied these cells with potential growth factors (in Groups 3 and 4). This led to the migration of porcine and human urothelial cells and a complete coverage of membranes with a size of up to 40 cm², with greater proliferation in a shorter space of time, without any spontaneous neoplastic transformation. The stimulation was possible only when 5% of bovine embryo serum (BES) was added and fibroblastic induction took place (in Groups 3 and 4) and was significantly higher than with exogenically completed media with 10% of bovine embryo serum (BES, in Group 1) or with epidermal growth factor (EGF) and all other additives (in Group 2).

This shows that culture media with fibroblast components contain active growth factors that are secreted in sufficiently high concentrations to compensate for the low concentration (5%) of bovine embryo serum (BES) or the absence of epidermal growth factor (EGF), bovine hypophysis extract (BHE), transferrin, hydrocortisone and insulin, added to the highly supplemented conventional culture media. These results also show that the mitogenic action and the signalling mechanism of the extracellular matrix (ECM) content of biological membranes and fibroblast-induced factors synergize each other. This favorable phenomenon can be used in optimized strategies to bring about the proliferation of the urothelium on biological matrices in vitro. Furthermore, the risk of infection transfer is minimized by the use of autologous fibroblasts instead of xenogenic feeder layers, i.e. xenogenic cell layers serving as growth substrates in the cell culture.

The increase in the mitotic rate and the delayed maturation of the urothelial cells were achieved in this study with a 5% bovine embryo serum (BES) supplement used in the first part of the urothelial technique. A further differentiation occurred when the amount of the bovine embryo serum (BES) was reduced (in Group 4). The bovine embryo serum (BES) concentration indeed inhibited the delayed proliferation of the cells and added the proliferative stage to the differentiation stage of the urothelium under stromal regulation and in the absence of other exogenically added growth factors. The proliferation of the cells was then regulated downward, which made terminal differentiation possible. This may be very important for the functionality of the urothelium and the inhibition of the hyperproliferation of cells after implantation. All four membranes exhibited terminally differentiated cells only in their surface layer, so that they resembled native tissues.

It has thus been found that the additional complex extracellular matrix (ECM) components, present in the membranes, combined with the stepwise adaptation of the medium, improved the conditions of culturing on these matrices in comparison with the fibroblast-conditioned cultures of urothelial cells on collagen gel [see refs. 10, 15]. It is a fact that the urothelial cell cultures on collagen gel cannot ensure the proliferation of the cells in more than three layers and cannot lead to terminal differentiation in a fibroblast-conditioned medium (FBCM) [see ref. 10], nor do they show any cellular expression for uroplakin III [see ref. 15].

In addition to its use in static cultures, the culture medium employed in Method 4 can be used for culturing in perfusion chambers and bioreactors when different media are chosen for special cell types that are cultured on both sides of the membrane. Similar studies on the induction of bladder muscle cells would be useful for the construction of complex live bladder transplants with highly differentiated cells, consisting of urothelial cells on one side, and smooth bladder muscle cells on the other side of the membrane. However, it is an important fact here that the terminally differentiated urothelial cells protect the transplanted material, i.e. the membrane with or without muscle cells, from urine.

In comparison with urothelial cells cultured in media with relatively expensive additives, the process according to the invention permits the proliferation and terminal differentiation of urothelial cells in a shorter space of time when culture media are used that do not contain these relatively expensive supplements. This favorable financial aspect is useful and should be taken into account in the preparation of bladder transplants.

The inducing action of the fibroblast-conditioned medium was observed not only on porcine urothelial cells but also on human ones (see FIG. 6).

FIG. 9 shows a schematic representation of the tissue transplant construct 1 according to the invention. The membrane 2 carries seven layers 3 of urothelial cells, with terminal differentiation of the urothelial cells in the outermost layer 5, while the other layers 4 consist of non-terminally differentiated urothelial cells.

The culturing conditions used for the urothelial cells according to the invention show a therapeutic potential for the induction of urothelium formation from a diseased organ.

Since the barrier properties of strongly differentiated urothelial constructs are the same as those of the native urothelium [see ref. 20], the culturing method according to the invention can be used to move a step closer to the ultimate aim of producing live and functionally autologous transplants with qualities similar to those of the native bladder.

Supplementation by expensive growth factors is not needed for making a construct according to the invention and for performing the process according to the invention, which is a considerable financial advantage. The process according to the invention enables one to prepare the construct according to the invention in a much shorter space of time than the conventional methods leading to similar constructs. This considerably reduces the time needed for the treatment of a patient whose urinary bladder is to be reconstructed. Not using gastrointestinal segments means that the complications associated with them can be avoided, and so further costs can be saved.

### LITERATURE

1. G. Falke, J. Caffaratti and A. Atala: “Tissue engineering of the bladder”, World J. Urol. 2000, 18, pp. 36-43
2. A. Atala: “New methods of bladder augmentation”, BJU Int. 2000, 85, pp. 24-34
3. A. Atala: “Bladder regeneration by tissue engineering”, BJU Int. 2001, 88, pp. 765-770
4. J. J. Yoo, J. Meng, F. Oberpenning and A. Atala: “Bladder augmentation using allogenic bladder submukosa seeded with cells”, Urology 1998, 51, pp. 221-225
5. Y. Zhang, B. P. Kropp, P. Moore et al.: “Coculture of bladder urothelial and smooth muscle cells on small intestinal submukosa: potential applications for tissue engineering technology”, J. Urol. 2000-, 164, pp. 928-934
6. E. Y. Cheng and B. P. Kropp: “Urologic tissue engineering with small intestinal submucosa: potential clinical applications”, World J. Urol. 2000, 18, pp. 26-30
7. R. Strehl, K. Schumacher, U. de Vries and W. W. Minuth: “Proliferating cells versus differentiated cells in tissue engineering”, Tissue Eng. 2002, 8, pp. 37-42
8. S. L. Voytik-Harbin, B. Rajwa and J. P. Robinson: “3D imaging of extracellular matrix and extracellular matrix-cell interactions”, Methods of Cell Biol. 2001, 63, pp. 583-597
9. M. Sittinger, J. Bujia, N. Rotter, D. Reitzel, W. W. Minuth and G. R. Burmester: “Tissue engineering and autologous transplant formation: practical approaches with resorbable biomaterials and new cell culture techniques”, Biomaterials 1996, 17, pp. 237-242
10. C. Fujiyama, Z. Masaki and H. Sugihara: “Reconstruction of the urinary bladder mucosa in three-dimensional collagen gel culture: fibroblast—extracellular matrix interactions on the differentiation of transitional epithelial cells”, J. Urol. 1995, 153, pp. 2060-2067
11. M. Erdani Kreft and M. Sterle: “The effect of lamina propria on the growth and differentiation of urothelial cells in vitro”, Pflugers Arch. 2000, 440, Suppl. 5, pp. R 181-182
12. S. D. Scriven, C. Booth, D. F. Thomas, L. K. Trejdosiewicz and J. Southgate: “Reconstruction of human urothelium from monolayer cultures”, J. Urol. 1997-, 158, pp. 1147-1152
13. A. Staack, I. Alexander, P. Merguerian and M. K. Terris: “Organ and species specificity in the stimulation of transitional epithelial cell growth by fibroblasts”, Euro. Urol. 2001, 39, pp. 471-477
14. W. I. de Boer, M. Vermeij, S. G. Diez de Medina et al: “Functions of fibroblasts and transforming growth factors in primary organoid-like cultures of normal human thelium”, Lab. Invest. 1996, 75, pp. 147-156
15. B. Ludwikowski, Y. Y. Zhang and P. Frey: “The long-term culture of porcine urothelial cells and induction of urothelial stratification”, BJU Int. 1999, 84, pp. 507-514
16. J. M. Rebel, W. I. de Boer, C. D. Thijssen, M. Vermey, E. C. Zwarthoff and T. H. van der Kwast: “An in vitro model of urothelial regeneration: effects of growth factors and extracellular matrix proteins”, J. Pathol. 1994, 173 (1994), pp. 283-291
17. N. C. Langkilde, H. Wolf and T. F. Orntoft: “Lectino-histochemistry of human bladder cancer: loss of lectin binding structures in invasive carcinomas”, APMIS 1989, 97, pp. 367-373
18. K. Lindberg and S. F. Badylak: “Porcine small intestinal submukosa (SIS): a bioscaffold supporting in vitro primary human epidermal cell differentiation and synthesis of basement membrane proteins”, Burns 2001, 27, pp. 254-266
19. K. A. R. Hutton, L. K. Trejdosiewicz , D. F. M. Thomas and J. Southgate: “Urothelial tissue culture for bladder reconstruction: an experimental study”, J. Urol. 1993, 150, pp. 721-725
20. S. Sugasi, Y. Leshros, I. Bisson, Y. Y. Zhang, K. Pavel and P. Frey: “In vitro engineering of human stratified urothelium: analysis of its morphology and function”, J. Urol. 2000, 164, pp. 951-957

1-31. (canceled)

32. A tissue transplant construct for the reconstruction of a human or animal organ, comprising:
a) a biologically compatible, collagen-containing membrane and
b) one or more layers of organ-specific tissue cells on the membrane,
where the outermost layer of the organ-specific tissue cells is a layer of terminally differentiated organ-specific tissue cells.

33. The tissue transplant construct according to claim 32, wherein the human or animal organ is a urinary bladder, and the organ-specific tissue cells are urothelial cells.

34. The tissue transplant construct according to claim 32, wherein the number of layers of tissue cells on the membrane is two to seven.

35. The tissue transplant construct according to claim 32, wherein the tissue cells are of human or porcine origin.

36. The tissue transplant construct according to claim 32, wherein the tissue cells are of autologous origin.

37. The tissue transplant construct according to claim 32, wherein the biologically compatible, collagen-containing membrane is a submucosal or dermal tissue.

38. The tissue transplant construct according to claim 32, wherein the biologically compatible, collagen-containing membrane is an intestinal submucosa, an acellular bladder submucosa or an acellular dermal matrix.

39. A method of forming a tissue transplant construct for reconstruction of a human or animal organ, the method comprising the steps of:
a) applying of one or more organ-specific tissue cells to a biologically compatible, collagen-containing membrane,
b) proliferating the applied tissue cells in a culture medium influenced by stromal induction and forming one or more layers of tissue cells on the membrane, and
c) terminal differentiating at least an outermost layer of the tissue cells, under influence of further stromal induction, by reducing an amount of mitogenic factors in the culture medium.

40. The method of forming the tissue transplant construct according to claim 39, wherein the amount of mitogenic factors in the culture medium is reduced to a level that is less than an original level of mitogenic factors in the culture medium when the step of proliferating the applied tissue cells commenced.

41. The method of forming the tissue transplant construct according to claim 39, wherein the tissue cells applied to the membrane in step a) are cultured in a medium that contains bovine embryo serum (BES) or autologous serum in order to bring about proliferation and terminal differentiation of the tissue cells.

42. The method of forming the tissue transplant construct according to claim 41, wherein the step of proliferating the applied tissue cells is effected in a culture medium that contains 5-10% of bovine embryo serum (BES) or autologous serum.

43. The method of forming the tissue transplant construct according to claim 41, wherein a concentration of the bovine embryo serum (BES) or the autologous serum is reduced in order to bring about terminal differentiation of the outermost layer.

44. The method of forming the tissue transplant construct according to claim 43, wherein the concentration of the bovine embryo serum (BES) or the autologous serum is reduced by at least 50%.

45. The tissue transplant construct according to claim 43, wherein the concentration of the bovine embryo serum (BES) or the autologous serum is reduced from 5% to 1%.

46. The method of forming the tissue transplant construct according to claim 43, wherein reduction of the bovine embryo serum (BES) or the autologous serum occurs when proliferation of the tissue cells applied to the membrane is so advanced that there are 3-7 layers of tissue cells on the membrane.

47. The method of forming the tissue transplant construct according to claim 43, wherein reduction of the bovine embryo serum (BES) or autologous serum occurs when the proliferation of the tissue cells applied to the membrane is so advanced that there are 4-7 layers of tissue cells on the membrane.

48. The method of forming the tissue transplant construct according to claim 39, wherein the tissue cells applied to the membrane in step a) are cultured in a fibroblast-conditioned medium (FBCM) that contains bovine embryo serum (BES) or autologous serum in order to bring about their proliferation and terminal differentiation.

49. The method of forming the tissue transplant construct according to claim 48, wherein the fibroblasts are obtained from the same individual as the tissue cells.

50. The method of forming the tissue transplant construct according to claim 39, wherein the organ-specific tissue cells are autologous urothelial cells and the culture medium is a fibroblast-conditioned medium (FBCM) that contains bovine embryo serum (BES).

51. The method of forming the tissue transplant construct according to claim 50, wherein the step of proliferating the applied tissue cells continues until 4-7 layers of urothelial cells have formed on the membrane.
